(19) United States
(12) Patent Application Publication
(10) Pub. No.: US 2022/0143128 A1
(43) Pub. Date: May 12, 2022

(54) METHODS FOR TREATING TRAUMATIC BRAIN INJURY

(71) Applicant: BCell Solutions, Inc., Colorado Springs, CO (US)

(72) Inventor: Martha Karen Newell-Rogers, Elizabeth, CO (US)

(73) Assignee: BCell Solutions, Inc., Colorado Springs, CO (US)

(21) Appl. No.: 17/291,271

(22) PCT Filed: Nov. 5, 2019
(86) PCT No.: PCT/US2019/059870

§ 371 Date: May 4, 2021

Related U.S. Application Data
(60) Provisional application No. 62/755,614, filed on Nov. 5, 2018.

(51) Int. Cl. A61K 38/03; A61K 31/365; C07K 16/24; A61P 25/00

(52) U.S. Cl. CPC A61K 38/03; A61P 25/00; C07K 16/24; A61K 31/365

(57) ABSTRACT

The invention relates to methods for treating traumatic brain injury by targeting specific innate and adaptive immune responses generated after the injury. The specific innate and adaptive immune responses may be targeted, for instance, using CLIP inhibitors, MIF antagonists and CD74 cleavage inhibitors and combinations thereof.

### RELATED APPLICATIONS

This application claims priority under 35 U.S.C. § 119(e) to U.S. provisional application No. 62/755,614, filed Nov. 5, 2018, the contents of which is incorporated herein by reference in its entirety.

### BACKGROUND OF INVENTION

Traumatic brain injury (TBI) can cause permanent damage to the brain, resulting in disability, emotional changes, epilepsy, chronic headaches, and other neurological dysfunction^{1,2}. Importantly, there are over 2 million TBIs annually and there is no effective therapy to prevent the serious consequences of brain trauma. TBI is an urgent problem, yet biomarkers, treatments, and mechanistic understanding are lacking for TBI pathogenesis and post-traumatic behavioral symptoms (PTBS).

### SUMMARY OF INVENTION

The invention in some aspects relates to the modulation of the immune response in the aftermath of traumatic brain injury (TBI). When a person suffers a TBI, there is an early inflammatory response and then some time later, some patients develop posttraumatic syndromes. These syndromes include cognitive impairment, as well as, learning, memory, attention, and emotional changes. It has been discovered that a protein, called CD74, contributes to the inflammatory response and post-traumatic syndromes after TBI. Methods for modulating CD74 activity and/or expression are used to improve outcomes of brain trauma patients in some aspects of the invention. Methods for identifying and carrying out new therapies based on the new drug targets that may be revealed from understanding how CD74 influences inflammation and nervous system damage after TBI are also provided.

The invention in some aspects is a method of treating a subject having a TBI, by administering to a subject having a TBI a macrophage migration inhibition factor (MIF) antagonist in an effective amount to treat the TBI. In some embodiments the TBI is post-traumatic brain injury syndromes (PTS) or post-traumatic behavioral symptoms (PTBS).

The invention in other aspects is a method of treating a subject having a TBI, by administering to a subject having a TBI a CD74 cleavage inhibitor in an effective amount to treat the TBI. In some embodiments the method further comprises administering to the subject an isolated MHC class II specific CLIP inhibitor. In other embodiments the method further comprises administering to the subject a MIF antagonist.

In some embodiments the MIF antagonist is an anti-MIF antibody. In some embodiments the anti-MIF antibody is a humanized anti-MIF antibody (Imalumab). In other embodiments the MIF antagonist is a small molecule MIF antagonist. The small molecule MIF antagonist may have one or more of the following properties: oral bioavailability and blood brain barrier permeability. In some embodiments the MIF antagonist is ISO1, MIF098, MIF139, MIF108, MIF046, MIFhom, and/or MIFacid.

In some embodiments the method further comprises administering to the subject an isolated MHC class II specific CLIP inhibitor. In some embodiments the CLIP inhibitor is synthetic. In other embodiments the CLIP inhibitor is a peptide or an siRNA. In yet other embodiments the CLIP inhibitor comprises a peptide having the sequence: X₁RX₂X₃X₄X₅LX₆X₇(SEQ ID NO: 1), wherein each X is an amino acid, wherein R is Arginine, L is Leucine and wherein at least one of X₂ and X₃ is Methionine, and wherein the peptide is a CLIP displacer. The peptide in some embodiments has any one or more of the following variables: X₁ is Phenylalanine; X₂ is Isoleucine; X₃ is Methionine; X₄ is Alanine; X₅ is Valine; X₆ is Alanine; and/or X₇ is Serine.

The peptide in some embodiments includes 1-5 amino acids at the N and/or C terminus. For instance, the peptide may have 1-5 amino acid at the C terminus of X₁RX₂X₃X₄X₅LX₆X₇(SEQ ID NO: 1) and/or the peptide may have 1-5 amino acid at the N terminus of X₁RX₂X₃X₄X₅LX₆X₇(SEQ ID NO: 1).

The peptide in other embodiments comprises FRIM X₄VLX₆S (SEQ ID NO: 3), wherein X₄ and X₆ are any amino acid. Optionally X₄ and X₆ are Alanine.

In some embodiments the peptide comprises FRIMAVLAS (SEQ ID NO: 4), IRIMATLAI (SEQ ID NO: 5), FRIMAVLAI (SEQ ID NO: 6), or IRIMAVLAS (SEQ ID NO: 7) or combinations thereof. The peptide in some embodiments has 9-20 amino acids.

In other embodiments the CLIP inhibitor comprises a peptide selected based on the subject's HLA-DR allele.

The method in some embodiments further comprises administering to the subject a CD74 cleavage inhibitor. In some embodiments the CD74 cleavage inhibitor is a SPP2La cleavage inhibitor. In other embodiments the SPP2La cleavage inhibitor is Brefeldin A. In some embodiments the CD74 cleavage inhibitor is a cathepsin S inhibitor. In other embodiments the cathepsin S inhibitor is Cystatin S, CST1, CST2, CST3 (cystatin C), CST4, CST5, CST6, CST7, CST8, CST9, CST11, CSTA (cystatin A), CSTB (cystatin B), histidine-rich glycoprotein (HRG), fetuins, cystatin-related protein, Spp24, cystatin-related epididymal spermatogenic (CRES) protein, Brefeldin A, Bortezomib (PS341), cystatin S, Z-FF-FMK, Z-FY-CHO, Z-FY(tBu)-DMK, E-64, E-64C, and/or E-64D.

The subject may be treated with the compounds of the invention at any time following a brain injury or TBI. For instance, the treatment may be immediately following an injury or perceived injury. For instance, the treatment may be within 5, 10, 20, 30, 40, or 50 minutes following the injury. In other embodiments the subject may be treated within 1 hour, 2, 3, 4, 5, 10, 15, 20, 24, 36, 72, or 120 hours following the TBI. In other embodiments the subject is treated within a week, 2 weeks, 1 month, 2 months, 6 months or 1 year of the TBI.

The subject may be, for instance, a military personnel or an athlete.

In other embodiments the method further involves using a head injury monitor to detect the presence of a head injury. The head injury monitor may be selected from the group consisting of a CheckLight™ device or a X-Patch™.

The subject is administered at least 2 doses of MIF antagonist in some embodiments. In other embodiments the subject is administered at least 3 doses MIF antagonist. In yet other embodiments the MIF antagonist is administered on a regular basis to the subject. For instance the MIF antagonist may be administered to the subject daily, every other day, or weekly.

In other aspects the invention is any of the compositions or combinations of compositions described herein for use in the treatment of a TBI or in the manufacture of a medicament for the treatment of TBI.

In other aspects, the invention is a method of treating a subject at risk of having a seizure, by administering to a subject at risk of having a seizure an isolated MHC class II specific CLIP inhibitor in an effective amount to treat the subject. In some embodiments the CLIP inhibitor is a synthetic peptide such as FRIMAVLAS (SEQ ID NO: 4).

In some embodiments the invention further comprises administering to the subject a CD74 cleavage inhibitor. In yet other embodiments the method further comprises administering to the subject a macrophage migration inhibition factor (MIF) antagonist such as an anti-MIF antibody. In some embodiments the anti-MIF antibody is a humanized anti-MIF antibody (Imalumab). In other embodiments the MIF antagonist is a small molecule MIF antagonist such as ISO1, MIF098, MIF139, MIF108, MIF046, MIFhom, or MIFacid.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a bar graph showing that at 24 hours after fluid percussion injury (FPI) in an animal model of TBI, there was decreased full-length cell surface CD74 on splenic B cells.

FIG. 2 is a bar graph showing changes in CLIP positive B cells in the brain after FPI in an animal model of TBI. ISO1 reduced the percent of B cells that express CLIP in the brain at 24 hrs after FPI.

FIGS. 3A-3C demonstrate that ISO1 has no effect on quantification of Fluorojade C (FJC) histology at 3 days after FPI in an animal model of TBI. FIG. 3A is a photomicrograph depicting FJC-staining in the peri-lesion area of a mouse treated with DMSO 30 mins after FPI. 3B is a comparative photomicrograph from a mouse treated with ISO1 at 30 mins after FPI. The data is depicted in a bar graph in 3C, demonstrating that ISO1 treatment had no effect on the total number of FJC-labeled cells in the perilesion area. N=6 per group.

FIGS. 4A-4D show GFAP-labeling in perilesion cortex of animal model of TBI at 3 days after FPI. FIG. 4A is a photomicrograph depicting GFAP+ astrocytes from an FPI mouse treated with DMSO vehicle at 30 mins after FPI. 4B is a representative photomicrograph from a mouse treated with ISO1 at 30 mins after FPI, showing a reduction in astrocyte response in the perilesion cortex. 4C is a photomicrograph demonstrating that CAP at 30 minutes after FPI had no significant effect on the astrocytic response to FPI. The data is depicted in a bar graph in 4D, demonstrating that ISO1 treatment significantly reduced GFAP-labeling compared to vehicle and compared to CAP in the perilesion area. The boxed areas are provided as inset enlargements to show the morphology of the astrocytes, some of the astrocytes from these mice still appear to be activated in response to FPI.

FIGS. 5A-5C is a set of bar graphs showing a dose response curve of inhibitors of various specific mechanisms of cleavage of full-length CD74 into CLIP 24 hours after FPI. 5A is a dose response curve for Brefeldin A at 30 minutes after FPI, demonstrating that Brefeldin A reduces cell surface CLIP on immune cells in the spleen, and that 5 mg/kg is the optimal dose to reduce CLIP after FPI. 5B is a dose response curve using the proteasomal inhibitor, Bortezomib (PS341), demonstrating that this compound was most efficacious at reducing cell-surface CLIP on B cells at 0.1 mg·kg, i.p. 5C is a dose response curve for Cystatin S, an inhibitor of Cathepsin S, which cleaves CD74 in B cells, dendritic cells, and macrophages, demonstrating that Cystatin-mediated reduced immune cell-surface CLIP following FPI, and that the optimal dosage is 50 mg/kg.

FIG. 6 is a set of bar graphs showing the overall number of leukocytes, T cells, CD4+ T cells, B cells and γδ T cells are all significantly increased in the brain after FPI. T-test, *P<0.001.

FIG. 7 is a set of bar graphs demonstrating that MHC class II expression is increased on splenocytes after FPI. In the left panel, the level of cell surface MHCII on splenocytes from naïve C57bl6 mice were significantly higher than naïve CD74def mice. In the right panel the levels of MHCII on splenocytes from CD74def mice showed a trend toward significant increases in MHCII after FPI. T-test, *P<0.001, *P=0.073. N=4 per group. FIGS. 8A-8F demonstrate early and chronic behavioral deficits after FPI are decreased in wild type mice treated with CAP, and in CD74Def mice. In FIG. 8A, neurological reflex testing was performed at 1-7 days after FPI. No deficit=0, moderate deficit=1 and severe deficit=2. On days 1 and 2, FPI mice exhibited moderately impaired pinna reflex. On days 1-6, FPI mice exhibited moderately impaired gate that was no longer evident by day 7. In FIG. 8B, FPI mice had a significantly longer (**P<0.005) righting time at 1 day after FPI. In FIG. 8C, there were no significant differences in locomotion in the open field task at 30 days after FPI. However, in FIG. 8D, FPI+vehicle mice spent significantly (*P<0.05) less time in the center of the open field at 30 days after FPI. This significant increase in anxiety after FPI was ameliorated in mice treated with CAP, or in CD74Def mice, suggesting that manipulating CD74 can improve these symptoms. In FIG. 8E, FPI resulted in a significant decrease (* P<0.05) in Novel Object Recognition at 30 days after injury. This effect was reversed in CAP-treated mice, and in CD74Def mice. In FIG. 8E, at 15 days post-FPI, FPI mice were deficient in novel arm exploration. This deficit was ameliorated by CAP treatment and in CD74Def mice. N=8 for all groups

FIGS. 9A-9C demonstrate that white matter is lost following FPI. DTI revealed significant alterations in white matter, particularly myelin at 30 days post FPI. In FIG. 9A, T2-weighted and relative anisotropy (RA) maps illustrated white matter abnormalities in the corpus callosum (CC) following FPI at the site of injury (*). Note the disrupted CC in the T2WI and RA color maps at the injury site. In FIG. 9B, Quantitative region of interest analysis from the CC on the side of injury in FPI mice (compared to shams) reveals no change in axial (axonal, p=0.464) but increased radial diffusivity (myelin, p=0.013) consistent with myelin degradation. RA, a measure of water asymmetry was also significantly reduced (p=0.006). Mean diffusivity was also increased but did not reach significance (p=0.075). Interestingly at this late time point, all the observed changes in WM integrity were bilateral, but often not as dramatic. In FIG. 9C, quantitative analysis of the width of CC, at 30 days after FPI revealed a reduced width of the ipsilateral CC. *P <0.005. (For A-C, Sham n=4, FPI n=6; in C, Naïve N=3).

FIG. 10 is a bar graph showing total splenic lymphocyte cells 1 day after FPI in male and female CD74def mice. In cells examined, no significant differences were seen between genders. (N=3 males).

### DETAILED DESCRIPTION

The invention, in aspects, involves new methods for treating brain disorders, such as brain injury. It has been discovered that specific innate and adaptive immune responses play a key role in the damaging effects of brain injury, and that these effects can be prevented, treated and/or reversed using the therapeutic strategies described herein.

Due to the nature of its direct impact on the brain, diagnosis and treatment of traumatic brain injury (TBI) has been mostly limited to treating the injury in the brain and central nervous system (CNS). Traumatic brain injury (TBI) is an urgent clinical problem, causing long-term and possibly permanent damage to the brain, and resulting in disability, emotional instability, epilepsy, chronic headaches, and other long-term neurological dysfunctions. Currently, there are no effective therapies to prevent the serious consequences of brain trauma and existing treatments that primarily target the CNS and/or neuroinflammation have not been successful. Contributions of both an innate and an adaptive immune response after TBI play a role in the development of these serious consequences.

CD74, a protein involved in both innate and adaptive immune responses, contributes to inflammation, neuroinflammation, and neurodegeneration following TBI. CD74 is a molecule involved in both innate and adaptive immune responses. The molecular mechanisms by which CD74 contributes to innate versus adaptive immunity are distinct, and each pathway contributes to distinct aspects of an immune response. Inflammatory events, including TBI, cause a rapid increase of pro-inflammatory cytokines, including macrophage migration inhibitory factor (MIF). When MIF binds to cell surface CD74 on B cells, a signaling cascade is initiated that results in an innate immune response, and the complex is internalized, lowering the level of CD74 on the B cell surface^{6,7}. After being internalized, the complex initiates downstream innate immune signaling, resulting from activation of the signal peptide peptidase-like 2a (SPPL2a) enzyme 89. Once activated, the SPPL2a enzyme cleaves CD74 into a 42-amino acid peptide, the N-terminal fragment (NTF), and a carboxy-terminal fragment^{8,9}. NTF serves as a transcription factor that stimulates NF-κB activity⁸. Stimulation of NF-kB promotes inflammation associated with an innate immune response^{10,11}. Thus, MIF-binding to full-length CD74 initiates innate immune signaling.

It has been discovered, in aspects of the invention, that the association of CD74 with MIF in the innate immune response after TBI, and the association of CD74 with MHCII in an adaptive immune response following TBI play important roles in the development of serious consequences associated with brain injury. Interestingly, antagonizing MIF, to interrupt the MIF-CD74 interaction reduced the typical astrocyte response to TBI, whereas disruption of the CD74-MHCII interaction reduced the secondary neurodegeneration that is observed 3 days after the initial injury. Moreover, based on characterization of the immune cell changes that occur in the spleen and the brain after TBI, a TBI-dependent expansion of B cells expressing the cleavage product of CD74, CLIP, in the groove of MHCII have been identified. Activation of CD4+ T cells after TBI have also been observed. These findings raise the interesting specter that B cells, via CD74, CLIP, and/or MHCII mechanisms, underlie the pathogenic response following TBI.

Although Applicant is not bound by mechanism, based, at least in part, on these observations, it is believed that CD74-specific contributions to the innate and adaptive immune responses following TBI can be targeted to improve post-traumatic behavioral syndromes (PTBS). Using a model of fluid percussion injury (FPI), the following phenomena are elucidated (1) the distinct contributions of full-length CD74, CLIPs, and MHCII on the innate and adaptive immune responses after FPI, (2) the impacts of these CD74-related mechanisms on neuropathology and behavioral outcomes, and (3) the pathogenic contribution of activated B and T cells following TBI.

Initially, it was determined that MIF-binding to CD74 impacts FPI-induced neuropathology and PTBS. As shown in the Examples section, the data generated in CD74^{def} mice demonstrated a role for CD74 in FPI-induced inflammation, neuroinflammation, and neurodegeneration, and on functional behavioral outcomes. MIF-binding to CD74 mediates pro-inflammatory, innate signaling by CD74. MIF antagonism was shown to disrupt MIF-full-length CD74 interactions and alter the pathogenic immune response to TBI.

The effects of CLIP on TBI-induced neuropathology and PTBS were shown to be MHC class II-dependent. Antagonizing CLIP-binding to MHCII limits neurodegeneration and immune cell activation/expansion after TBI. Because it is possible that the effects of CLIP are dependent on binding to the antigen-binding groove of MHCII, therapeutic strategies targeting either the cleavage of CD74 into CLIP or antagonizing CLIP-binding to MHC class II may be used.

Additionally the data has demonstrated that CD74 or CLIP-expressing B cells are pathogenic contributors to TBI. B cells are capable of activating T cells and producing auto-antibodies, and human studies have demonstrated brain-specific antibodies after TBI, indicating that an adaptive immune response has occurred in response to TBI. The data presented in the Examples indicate that a synthetic antigenic peptide that antagonizes CLIP-binding to MHC class II (CLIP inhibitor) is neuroprotective, anti-inflammatory, and reduces the expansion of B cells and T cells following TBI. Both the acute astrocyte and microglial activation and the exacerbation caused by later insult may be reversed, by administering the small peptide (CLIP inhibitor) predicted to selectively eliminate pro-inflammatory immune cells. Thus, the MIF antagonists may be administered with a CLIP inhibitor in order to provide additional therapeutic benefit.

Thus, the invention involves several important discoveries. For instance, it was found that CD74, and its cleavage products, contribute to TBI-dependent neuropathology and TBI-dependent changes in neurobehavior, via distinct mechanisms that modulate the innate and adaptive immune responses following TBI. Thus, contributions of CD74 to the innate and adaptive immune responses following TBI can be selectively targeted to improve post-traumatic outcomes.

The terms “traumatic brain injury” or “TBI” are used herein to refer to syndromes or pathologies associated with brain injury. Acute brain injury disrupts the normal function of the brain and generally has a poor prognosis for functional recovery and survival. The effects of TBI can be severe, including severe neurocognitive, physical, and psychosocial impairment. Treatment for TBI can comprise reducing or preventing further neurodegeneration. As described further below, a 10-fold reduction was observed in a mouse model. In some embodiments, treating traumatic brain injury involves reducing gliosis or treating the secondary effects of TBT. Representative secondary effects include for instance reperfusion injury, delayed cortical edema, blood-brain barrier breakdown, local electrolyte imbalance, neurovascular unit dysfunction, and intracranial pressure.

TBI may result from a physical brain injury event or a internal response such as a brain injury caused by stroke or hypoxia. The resultant TBI may be a mild TBI, medium TBI, or severe TBI, with symptoms dependent on the severity of the trauma. For example, a mild traumatic brain injury may cause the injured person to experience physical symptoms such as loss of consciousness or nausea. However, moderate or severe traumatic brain injury, may cause an injured person to fall into a coma or a vegetative state, or experience seizures, fluid buildup in the cerebral ventricles and/or blood vessel damage, leading to nerve damage, cognitive problems, issues with problem-solving and organization skills as well as social and behavioral problems.

CD74 is well characterized for its contribution to antigen processing and presentation, via antigen presenting cells (APCs), including B cells, that express Major Histocompatibility Complex class II (MHCII)¹⁵. Full-length CD74 serves as a chaperone for MHCII transport from the golgi and endoplasmic reticulum to the lysosome where antigen processing occurs, and in the transport of MHCII to the cell surface¹⁵. In the lysosome, CD74 is cleaved by Cathepsin S in APCs, primarily B cells, resulting in the formation of CLIP. In the lysosome, CLIP then occupies the antigen-binding groove of MHCII until HLA-DM (H-2M in mice) catalyzes the replacement of CLIP with antigenic peptides. In this cleaved form, CD74 facilitates the transition to an adaptive immune response via the processing and presentation of antigenic peptides associated with MHCII^{4,16-18}.

The discovery that the adaptive and innate immune responses play a role in exacerbating the cognitive and behavioral consequences of TBI has important implications for the treatment of these disorders. A method of treating a subject having a TBI by administering an effective amount to treat the subject of a MIF antagonist is provided.

The novel use of selective MIF inhibitors in order to inhibit the pro-inflammatory innate signaling by CD74 resulting from TBI is an important component in the treatment of this type of injury. MIF is an endogenous ligand for CD74 that initiates a cellular signaling cascade by recruiting cell surface CD44. A number of MIF antagonists have been developed and several have been tested in humans. For instance, humanized anti-MIF (Imalumab) has been tested in humans (completed phase II clinical testing) for the inhibition of CD74-dependent signal transduction. Additionally small molecule MIF antagonists are in advanced pre-clinical development. Among the MIF antagonists that are useful are molecules with advantageous pharmacologic properties, including oral bioavailability and blood brain barrier permeability.

Macrophage migration inhibitory factor (MIF) is a cytokine that is a critical regulator of the innate and adaptive immune response and is a necessary factor for the activation or proliferative responses of macrophages. MIF is released by a variety of cell types, proceeding via an autocrine/paracrine activation pathway involving the p44/p42 (ERK-1/2) mitogen-activated protein kinase cascade. MIF binds to the extracellular domain of Ii, a Type II membrane protein, and causes signaling through the extracellular signal-related kinase (ERK)-1/2MAP kinase cascade and cell proliferation.

A MIF antagonist, as used herein, is a compound that competes with MIF for binding to the Ii (CD74) polypeptide or otherwise inhibits the interaction of the MIF with the Ii (CD74) polypeptide. MIF antagonists include protein, small molecule and nucleic acid compounds. Many MIF antagonists are known to the skilled artisan. Others may be identified using assays such as binding assays, i.e., an assay may be conducted with recombinantly prepared MIF and Ii peptides, one of which is optionally immobilized to a solid support, and one of which (or a binding partner thereto, such as an antibody) is labeled to facilitate detection and measurement of the MIF:Ii binding interaction.

In some embodiments the MIF antagonist may be a protein. For instance, the MIF antagonist may be a peptide that binds to MIF, blocking its interaction with CD74. The protein may be an antibody or fragment thereof.

In some embodiments the anti-MIF antibody is imalumab or fragments thereof. Imalumab is a monoclonal IgG1-kappa antibody that upon intravenous administration, binds to MIF, blocking its activity and preventing the MIF-mediated secretion of certain cytokines. Imalumab, also known as BAX69, is an antibody against MIF which is commercially available from Creative Biolabs and has an amino acid sequence as presented in UniProt P14174 (MPMFIVNTNV PRASVPDGFL SELTQQLAQA TGKPPQYIAV HVVPDQLMAF GGSSEPCALC SLHSIGKIGG AQNRSYSKLL CGLLAERLRI SPDRVYINYY DMNAANVGWN NSTFA) (SEQ ID NO. 8). Numerous other monoclonal and polyclonal antibodies are commercially available and may be used in the methods described herein or used to produce humanized antibodies or scFv.

In other embodiments the MIF antagonist may be a small molecule. For instance the small molecule MIF antagonist may be a compound described in US Patent Publication No. 2018/0162813, U.S. Pat. No. 7,378,416, and US Patent Publication No. 2009-0130165. U.S. Pat. No. 7,378,416 discloses 3,4-dihydro-benzo[e][1,3]oxazin-2-ones which are substituted at the nitrogen atom by unsubstituted or substituted (C3-8)cycloalkyl, (C1-4)alkyl(C3-8)cycloalkyl, (C6-18)aryl or (C6-18)aryl(C1-4)alkyl. US Patent Publication No. 2009-0130165 discloses MIF-inhibiting benzimidazolone analogues and derivatives.

US Patent Publication No. 2018/0162813 discloses MIF modulators useful according to the invention that are bicyclic compounds according to the chemical structure (I):

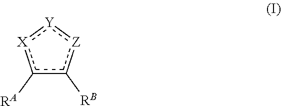

where X is O, S, N—RN^{XN1} or CR^{XC1}R^{XC2}; Y is N—R^{YN1} or CR^{YC1}R^{YC2}; and Z is O, S, N—R^{ZN1} or CR^{ZC1}R^{ZC2}, with the proviso that at least one of X or Z is N—R^{YN1} and X and Z are other than O, when Y is O; R^{XN1} is absent (N is —N═, thus forming a double bond with an adjacent atom), H or an optionally substituted C₁-C₈ alkyl, alkene or alkyne group, an optionally substituted C₁-C₇ acyl group, an optionally substituted (CH₂)j-phenyl group or an optionally substituted (CH₂)ₘ-heterocyclic (preferably heteroaryl) group, or an optionally substituted carbonyl phenyl group, or an optionally substituted carbonyl heteroaryl group and the R groups as defined in US Patent Publication No. 2018/0162813, which is hereby incorporated by reference for each MIF antagonist disclosed therein.

Some preferred MIF antagonists include but are not limited to MIF098 and metabolites thereof and analogs thereof, such as the compounds shown in the following structures:

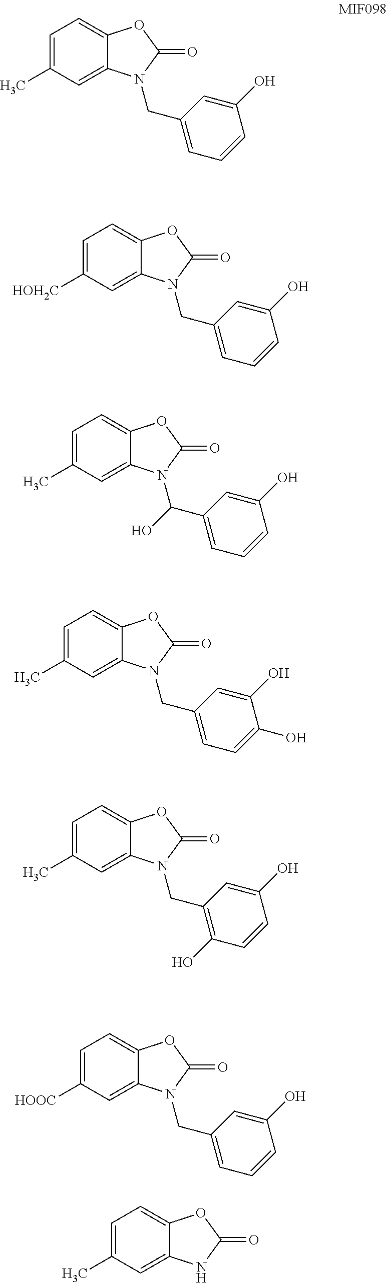

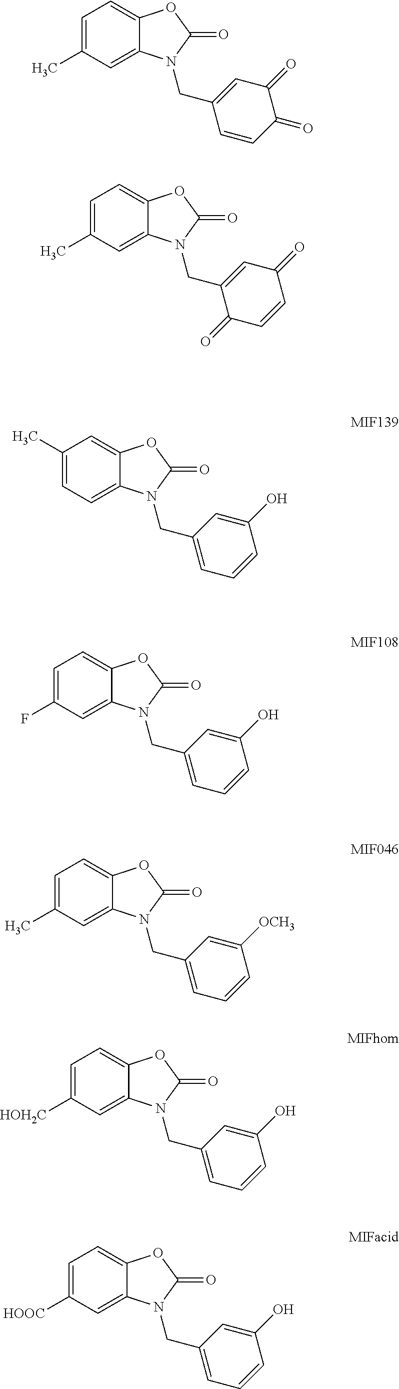

Each of the compounds disclosed herein includes a pharmaceutically acceptable salt, stereoisomer (e.g. enantiomer or diastereomer), solvate or polymorph thereof.

In some embodiments the small molecule MIF antagonist is ISO1. ISO1 is a commercially available potent, cell-permeable orally bioavailable MIF antagonist that targets the catalytic pocket of MIF and inhibits TNF release from macrophages (Abcam, Tocris). ISO1 is also known as 4,5-Dihydro-3-(4-hydroxyphenyl)-5-isoxazoleacetic acid methyl ester, having a molecular weight of 235.24 and the following chemical structure:

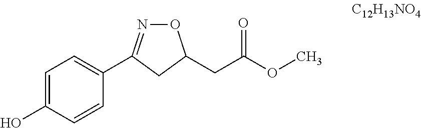 [SMILES: COC(=O)CC1CC(c2ccc(O)cc2)=NO1]

The MIF therapy of the invention can be combined with methods for immune cell depletion using CLIP inhibitors (i.e. a death-inducing peptide) or by therapeutic use of selective immune cell depletion using highly specific therapeutic antibodies as treatments for brain injury resulting from initial neuronal damage. A number of small amino acid peptides that are predicted to bind in the groove of MHCII alleles with a greater binding constant than the invariant MHC-associated peptide, CLIP, are useful in this aspect of the invention. These CLIP inhibitors can target pro-inflammatory, MHCII-expressing immune cells by causing MHCII-mediated death of pro-inflammatory antigen presenting cells. MHCII-mediated cell death has been described as a part of T cell recognition resulting in both T cell activation and the death of antigen presenting cells. Such immune cells are implicated in detrimental immune responses in the CNS, culminating in permanent loss of neurons following traumatic brain injury. These peptides or depleting antibodies can be used to eliminate the immune cells.

Thus, the invention in some aspects involves the use of a MIF antagonist with another compound such as a CLIP inhibitor or a CD74 cleavage inhibitor for treating the TBI. A CLIP inhibitor as used herein is any molecule that reduces the association of a CLIP molecule with MHC, for instance, by binding to the MHC and blocking the CLIP-MHC interaction or inhibiting the expression of CLIP. The CLIP inhibitor may function by displacing CLIP from the surface of a CLIP molecule expressing cell. A CLIP molecule expressing cell is a cell that has MHC class I or II on the surface and includes a CLIP molecule within that MHC.

The CLIP molecule, as used herein, refers to intact CD74 (also referred to as invariant chain) or intact CLIP, as well as the naturally occurring proteolytic fragments thereof. Intact CD74 or intact CLIP refer to peptides having the sequence of the native CD74 or native CLIP respectively. The CLIP molecule is one of the naturally occurring proteolytic fragments of CD74 or CLIP in some embodiments. The CLIP molecule may be, for example, at least 90% homologous to the native CD74 or CLIP molecules. In other embodiments the CLIP molecule may be at least 91%, 92%, 93%, 94%, 95%, 96%, 97%, 98%, or 99% homologous to the native CD74 or CLIP molecules. An example of native CLIP molecule is MRMATPLLM (SEQ ID NO: 2), and in three-letter abbreviation as: Met Arg Met Ala Thr Pro Leu Leu Met (SEQ ID NO: 2).

CLIP inhibitors include peptides and small molecules that can replace CLIP. In some embodiments the CLIP inhibitor is a peptide. A number of peptides useful for displacing CLIP molecules are described in U.S. patent application Ser. No. 12/508,543 (publication number US-2010-0166782-A1); Ser. No. 12/739,459 (publication number US-2011-0118175) and Ser. No. 12/508,532 (publication number US-2010-0166789-A1) each of which is herein specifically incorporated by reference.

CLIP inhibitors include for instance but are not limited to competitive CLIP fragments, MHC class II binding peptides and peptide mimetics. Thus, the CLIP inhibitor includes peptides and peptide mimetics that bind to MHC class II and displace CLIP. For instance, an isolated peptide comprising X₁RX₂X₃X₄X₅LX₆X₇ (SEQ ID NO: 1), wherein each X is an amino acid, wherein R is Arginine, L is Leucine and wherein at least one of X₂ and X₃ is Methionine. X refers to any amino acid, naturally occurring or modified. In some embodiments the Xs referred to the in formula X₁RX₂X₃X₄X₅LX₆X₇ (SEQ ID NO: 1) have the following values:

X₁ is Ala, Phe, Met, Leu, Ile, Val, Pro, or Trp

X₂ is Ala, Phe, Met, Leu, Ile, Val, Pro, or Trp

X₃ is Ala, Phe, Met, Leu, Ile, Val, Pro, or Trp.

wherein X₄ is any

X₅ is Ala, Phe, Met, Leu, Ile, Val, Pro, or Trp

X₆ is any

X₇ is Ala, Cys, Thr, Ser, Gly, Asn, Gln, Tyr.

The peptide preferably is FRIM X₄VLX₆S (SEQ ID NO: 3), such that X₄ and X₆ are any amino acid and may be Ala. Such a peptide is referred to as FRIMAVLAS (SEQ ID NO: 4), also referred to as TPP. Other preferred peptides include: IRIMATLAI (SEQ ID NO: 5), FRIMAVLAI (SEQ ID NO: 6), and IRIMAVLAS (SEQ ID NO: 7).

The minimal peptide length for binding HLA-DR is 9 amino acids. However, there can be overhanging amino acids on either side of the open binding groove. For some well-studied peptides, it is known that additional overhanging amino acids on both the N and C termini can augment binding. Thus the peptide may be 9 amino acids in length or it may be longer. For instance, the peptide may have additional amino acids at the N and/or C terminus. The amino acids at either terminus may be anywhere between 1 and 100 amino acids. In some embodiments the peptide includes 1-50, 1-20, 1-15, 1-10, 1-5 or any integer range there between. When the peptide is referred to as “N-FRIMAVLAS-C” (SEQ ID NO: 4) or “N-X₁RX₂X₃X₄X₅LX₆X₇-C” (SEQ ID NO: 1) the -C and -N refer to the terminus of the peptide and thus the peptide is only 9 amino acids in length. However the 9 amino acid peptide may be linked to other non-peptide moieties at either the -C or -N terminus or internally.

The peptide may be cyclic or non-cyclic. Cyclic peptides in some instances have improved stability properties. Those of skill in the art know how to produce cyclic peptides.

The peptides may also be linked to other molecules. The peptide and molecule may be linked directly to one another (e.g., via a peptide bond); linked via a linker molecule, which may or may not be a peptide; or linked indirectly to one another by linkage to a common carrier molecule, for instance.

Thus, linker molecules (“linkers”) may optionally be used to link the peptide to another molecule. Linkers may be peptides, which consist of one to multiple amino acids, or non-peptide molecules. Examples of peptide linker molecules include glycine-rich peptide linkers (see, e.g., U.S. Pat. No. 5,908,626), wherein more than half of the amino acid residues are glycine. Preferably, such glycine-rich peptide linkers consist of about 20 or fewer amino acids.

The peptide for instance, may be linked to a PEG or TEG molecule. Such a molecule is referred to as a PEGylated or TEGylated peptide.

In certain embodiments, the CLIP inhibitor is an inhibitory nucleic acid such as a small interfering nucleic acid molecule such as antisense, RNAi, or siRNA oligonucleotide to reduce the level of mature CLIP molecule (CD74) expression. The nucleotide sequences of CD74 molecules are well known in the art and can be used by one of skill in the art using art recognized techniques in combination with the guidance set forth herein to produce the appropriate siRNA molecules.

Small interfering nucleic acid (siNA) include, for example: microRNA (miRNA), small interfering RNA (siRNA), double-stranded RNA (dsRNA), and short hairpin RNA (shRNA) molecules. An siNA useful herein can be unmodified or chemically-modified. An siNA can be chemically synthesized, expressed from a vector or enzymatically synthesized. Such methods are well known in the art. Exemplary single stranded regions of siRNA for CLIP include: GGUAGUAAUUAGAACAAAA (SEQ ID NO: 9); GGUUCACAUUAGAAUAAAA (SEQ ID NO: 10); GAACAAAAAAAAAAAAAAA (SEQ ID NO: 11); CAAAAAAAAAAAAAAAAAA (SEQ ID NO: 12); AGAACAAAAAAAAAAAAAA (SEQ ID NO: 13); ACAAAAAAAAAAAAAAAAA (SEQ ID NO: 14); GUAAUUAGAACAAAAAAAA (SEQ ID NO: 15); CAUGGUUCACAUUAGAAUA (SEQ ID NO: 16); GUAGUAAUUAGAACAAAAA (SEQ ID NO: 17); and GGCUUUUCUAGCCUAUUUA (SEQ ID NO: 18). Others are contemplated as well.

In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

Other inhibitor molecules that can be used include ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9,1996). Multitarget ribozymes (connected or shotgun) have been suggested as a means of improving efficiency of ribozymes for gene suppression (Ohkawa et al., Nucleic Acids Symp Ser. (29):121-2, 1993).

CD74 is a cell surface receptor for MIF and binding of MIF to CD74 induces intramembrane cleavage of CD74 and release of its cytosolic intracellular domain (ICD), which regulates cell survival. One of the initial steps of an antigen-specific T cell response to external antigens, is the formation of peptide-MHC class II complexes in antigen presenting cells in which, cysteine proteases play a key role in degrading the proteins within the endosomal-lysosomal compartments of APCs and in the cleavage of MHC class II-associated invariant chain, li, leading to the formation of clip associated MHC-molecules.

A CD74 cleavage inhibitor, as used herein, is a compound that prevents or reduces the amount of CD74 cleavage and thus release of ICD. Exemplary cleavage inhibitors include but are not limited to inhibitors (the cystatins) of cathepsin proteases. The cathepsin family contains several classes of proteases. For instance, the cysteine protease class comprises cathepsins B, L, H, K, S, and O. The aspartyl protease class is composed of cathepsins D and E. Cathepsin G is in the serine protease class. Cystatins that inhibit each of these protease classes are useful in the methods of the invention. For example Cathepsin S inhibitors, Cathepsin L inhibitors, Cathepsin B inhibitors, and Cathepsin H inhibitors are all included within the methods of the invention.

Cystatins are the reversible competitive inhibitors of C1 cysteine proteases. The cystatins are grouped into three major families. 1) The stefins include at least stefin A and B, which are also known as cystatin A and B. The stefins are unglycosylated inhibitors that are generally expressed intracellularly. 2) Family 2 (cystatins) have molecular masses in the range of 13-14 kDa, and are represented by cystatin C, D, S, SA, and SN. 3) The kininogens have molecular weights in the range of 88-114 kDa, are glycosylated and have 2 domains (domains 2 and 3) which have protease inhibitory activities. Cystatins include but are not limited to CST1, CST2, CST3 (cystatin C, a marker of kidney function), CST4, CST5, CST6, CST7, CST8, CST9, CST11, CSTA (cystatin A), CSTB (cystatin B), histidine-rich glycoprotein (HRG), fetuins, cystatin-related protein, Spp24, cystatin-related epididymal spermatogenic (CRES) protein, Brefeldin A, Bortezomib (PS341), cystatin S, Z-FF-FMK, Z-FY-CHO, Z-FY(tBu)-DMK, E-64, E-64C, and E-64D.

In some aspects the invention is a method for treating a subject having or at risk of having a seizure or epilepsy. It has been demonstrated herein that cell surface CLIP on B cells plays a role in seizure severity and mortality in an animal model. It was demonstrated that peptide CLIP inhibitor treatment after pilocarpine (in the animal model) reduced mortality and decreased seizure severity, consistent with the hypothesis that seizure involves components of antigen processing and presentation, and more specifically CLIP+ B cells. Thus, the results disclosed herein provide a foundation for targeting CLIP+ B cells for the treatment of seizures and forms of epilepsy that are known to involve the adaptive immune system, such as auto-immune epilepsies. A subject at risk of having a seizure or epilepsy is a subject that has previously had a seizure or has been diagnosed as having seizure or epilepsy risk.

The invention involves methods for treating a subject. A subject shall mean a human or vertebrate mammal including but not limited to a dog, cat, horse, goat and primate, e.g., monkey. Thus, the invention can also be used to treat brain injury in non-human subjects. Preferably the subject is a human. In some embodiments the subject has a traumatic brain disorder.

As used herein, the term treat, treated, or treating when used with respect to a disorder refers to a treatment after the subject has developed the injury in order to prevent the consequences of brain injury, prevent the damage from becoming worse, or slow the progression of the damage compared to in the absence of the therapy.

When used in combination with the therapies of the invention the dosages of known therapies may be reduced in some instances, to avoid side effects.

When administered in combination with other therapeutic agents the CLIP inhibitor may be administered simultaneously or sequentially. When the other therapeutic agents are administered simultaneously they can be administered in the same or separate formulations, but are administered at the same time. The administration of the other therapeutic agent and the CLIP inhibitor can also be temporally separated, meaning that the therapeutic agents are administered at a different time, either before or after, the administration of the CLIP inhibitor. The separation in time between the administration of these compounds may be a matter of minutes or it may be longer.

The active agents of the invention are administered to the subject in an effective amount for treating disorders such as neurological disorders. An “effective amount”, for instance, is an amount necessary or sufficient to realize a desired biologic effect. An effective amount for treating neurological disorders may be an amount sufficient to reduce neurological deficits and/or to reduce blood brain barrier permeability and/or to reduce circulating peripheral cells. According to some aspects of the invention, an effective amount is that amount of a compound of the invention alone or in combination with another medicament, which when combined or co-administered or administered alone, results in a therapeutic response to the disease, either in the prevention or the treatment of the disease. The biological effect may be the amelioration and or absolute elimination of symptoms resulting from the disease. In another embodiment, the biological effect is the complete abrogation of the disease, as evidenced for example, by the absence of a symptom of the disease.

The effective amount of a compound of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

In particular a number of the autophagy and fatty acid metabolism inhibitors described herein have been safely administered to humans. Safe doses for chronic or acute therapies of these compounds are known to the skilled artisan. For example chloroquine and hydroxychloroquine have been chronically administered to humans for the treatment of malaria infection as well as some forms of autoimmune disease. Dichloroacetate (DCA) has been administered to subjects for the treatment of metabolic disorders. Chronic therapy with these compounds at doses effective for inhibiting autophagy have proven to be safe in long term administration protocols. Chloroquine typically is administered in a dosage of 300 mg-600 mg to adults for the treatment of malarial infection. DCA can be used, for example, in dosages of 1-25 mg/kg of body weight per day, 1-15 mg/kg of body weight per day, or 5-10 mg/kg of body weight per day.

Multiple doses of the molecules of the invention are also contemplated. In some instances, when the molecules of the invention are administered with another therapeutic, a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The compounds of the invention, in some aspects, may be formulated in a device for individual delivery. For instance, the device may be similar to an epipen. The device may have a housing connected to a needle and a spring-loaded mechanism for a single delivery of a predetermined amount of active agent. The advantage of these devices is that they can be used by an individual who has experienced a brain injury, very close in time to the injury. For instance, military personnel who are at risk of head injuries, or athletes competing in sports that are associated with the risk of head injury can maintain such a device.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier. Pharmaceutically-acceptable carriers for peptides, monoclonal antibodies, and antibody fragments are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Therapeutic formulations of the compounds, i.e., peptides, small molecules, nucleic acids or antibodies may be prepared for storage by mixing a compounds having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The compounds may be administered directly to a cell or a subject, such as a human subject alone or with a suitable carrier. Additionally, a peptide may be delivered to a cell in vitro or in vivo by delivering a nucleic acid that expresses the peptide to a cell. Various techniques may be employed for introducing nucleic acid molecules into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. For certain uses, it is preferred to target the nucleic acid molecule to particular cells. In such instances, a vehicle used for delivering a nucleic acid into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half-life, and the like. Polymeric delivery systems also have been used successfully to deliver nucleic acid molecules into cells, as is known by those skilled in the art. Such systems even permit oral delivery of nucleic acid molecules.

Peptide or nucleic acid therapeutics may also be delivered to mammalian cells using a mammalian expression vector. Such a vector can be delivered to the cell or subject and the peptide expressed within the cell or subject. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the a-fetoprotein promoter.

As used herein, a “vector” may be any of a number of nucleic acid molecules into which a desired sequence may be inserted by restriction and ligation for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of neurological disorders.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1: CD74 Contributes to the Innate and Adaptive Immune Responses Following TBI and Therapeutics that Modulate CD74 Influence the Progression of TBI

The contributions of full-length cell-surface CD74 and its proteolytic products (CLIPs) in the pathogenic mechanisms that follow TBI were assessed. First, we determined if full-length CD74 on B cells was altered after fluid percussion injury (FPI) in an FPI model of TBI. A mouse model of TBI was produced and splenoctyes from those mice were isolated following FPI. The model, referred to as the “fluid percussion injury model”, is referenced in Tobin et al Acta Neuropathologica Communications 2014. Splenocytes were stained with anti-CD19 for B cells and counterstained with anti-CD74.

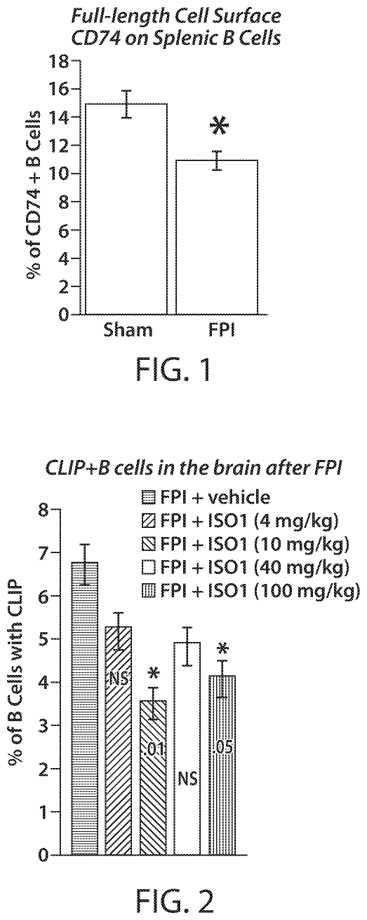

The data demonstrates that at 24 hours after FPI, there was a significant decrease in full-length cell surface CD74 on splenic B cells (FIG. 1). The reduction of CD74 after FPI indicates either that CD74 has been internalized and/or that CD74 has been cleaved into CLIPs.

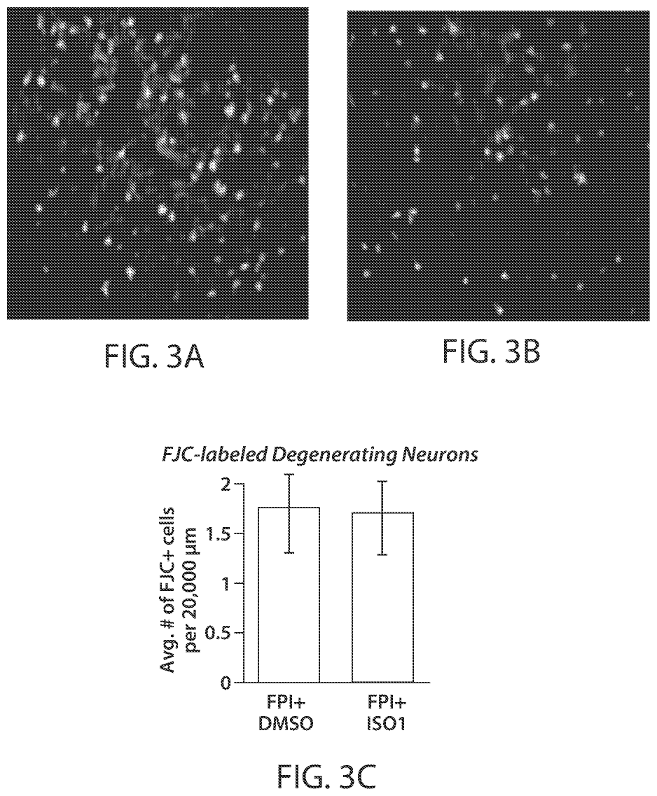

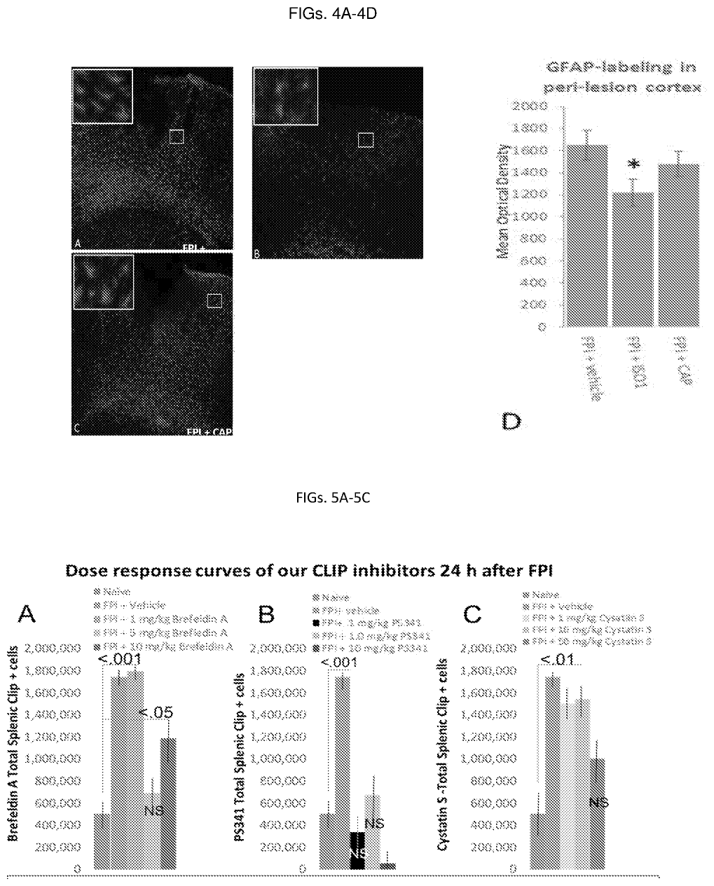

In order to address the innate immune components of CD74, a series of experiments in which MIF-binding to CD74 was antagonized at 30 min after FPI were performed. MIF mRNA levels are elevated after TBI. This occurs as a part of the innate immune response, via MIF-binding to, and signaling through cell surface CD74³⁵. We utilized ISO1, a small molecule that inhibits MIF-binding to CD74, to determine whether inhibition of MIF-binding would alter FPI-induced, CLIP-related cellular (FIG. 2) and neuroanatomical outcomes (FIGS. 3 and 4). ISO1 was administered at 30 mins after FPI and performed brain flow cytometry to assess what percent of leukocytes are CLIP+ B cells. It should be noted that less than 0.001 B cells enter the brain in naïve mice so they are not included in the graph. We found that ISO1 administration at 30 min after FPI significantly reduced the percentage of CLIP+ B cells in the brain (FIG. 2), largely in a dose dependent manner. The results show that a 10 mg/kg dose of ISO1 at 30 mins after FPI provided the most significantly robust decrease in the percent of CLIP+ B cells entering the brain.

We assessed the effects of 10 mg/kg of ISO1 administered at 30 mins after FPI on neurodegeneration (FIG. 3), and the astrocytic response (FIG. 4), in peri-injury cortex, at 3 days after FPI. We found that ISO1 had no effect on neurodegeneration (FIG. 3), but significantly reduced the peak astrocyte response at 3 days post-FPI (FIG. 3). Conversely, administration of CAP, which blocks CLIP-binding to MHCII, significantly ameliorates neurodegeneration after FPI¹⁷, whereas administration of CAP had no significant effect on the astrocytic response to FPI (FIG. 3C).

Therefore, inhibition of MIF-binding to CD74, the innate signaling component of CD74, results in a decrease in astrocyte activation, but not neurodegeneration. Conversely, inhibition of CLIP-binding to MHCII, an important step in the initiation of an adaptive immune response, has no effect on astrocyte activation after FPI, but does reduce neurodegeneration. These data suggest that CLIP+ B cells, via their role in MIF-mediated innate immune signaling, or CLIP-mediated adaptive immune signaling are involved in several pathogenic responses to TBI.

In addition to inhibiting MIF-binding to CD74, we have reduced the level of CLIP by inhibiting the distinct mechanisms by which CD74 is cleaved. Drugs that inhibit CD74 cleavage have been administered 30 min after FPI and the levels of CLIPs in the spleen have been assessed at 1 day post-FPI. Our data show that inhibition of SPP2La cleavage of CD74 by Brefeldin A reduces cell surface CLIP on immune cells in the spleen, and that 5 mg/kg is the optimal dose to reduce CLIP after FPI (FIG. 5A). We also found, using the proteasomal inhibitor, Bortezomib (PS341), that this compound was most efficacious at reducing cell-surface CLIP on B cells at 0.1 mg·kg, i.p. (FIG. 5B). Finally, Cystatin S is an inhibitor of Cathepsin S, which cleaves CD74 in B cells, dendritic cells, and macrophages^{11,12}. Treatment with Cystatin S reduces the level of CLIPs in MHCII³⁶. The data presented herein demonstrate a Cystatin-mediated reduction in immune cell-surface CLIP following FPI. The optimal dosage is 50 mg/kg (FIG. 5C). At concentrations greater than 50 mg/kg, the compound appeared to be toxic under the tested conditions. The optimal dose for each of these compounds, that safely inhibit each of the mechanisms by which CD74 is cleaved into CLIPs after FPI have been identified.

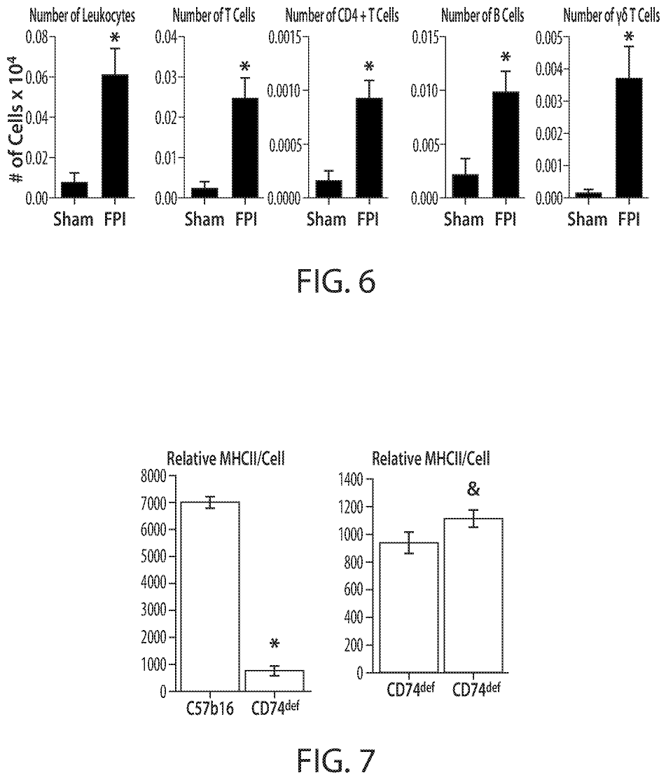

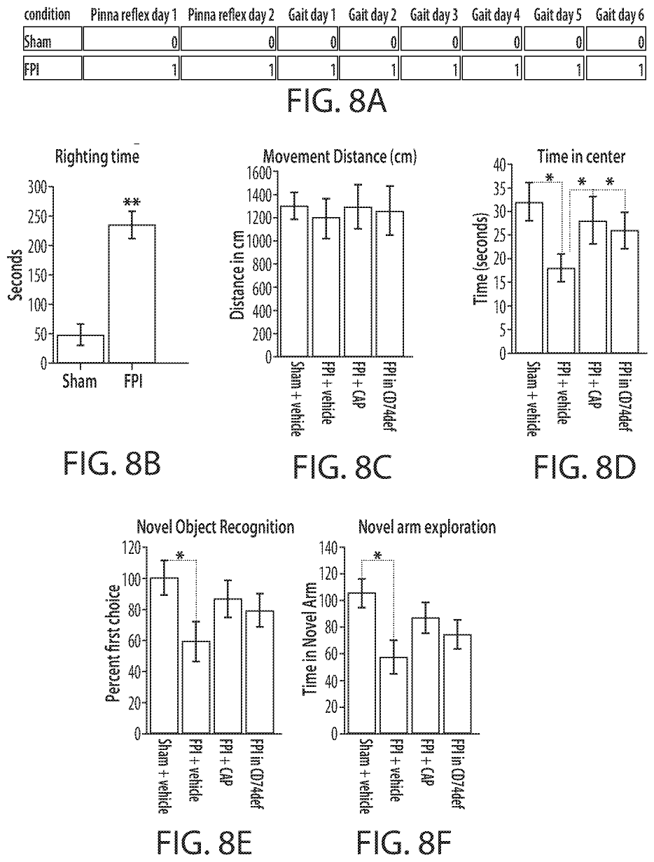

The type and extent of brain infiltrating immune cells was determined after FPI. At 24 hours after FPI, a significant increase in the number of leukocytes, including CD3+ T cells, CD4+ T cells, 78 T cells, and B cells was observed following injury (FIG. 6). The expansion and migration of B cells to sites of inflammation may enhance the likelihood of antigen processing and presentation of self-antigens by the newly expanded B cells, thereby facilitating the recognition of self-antigens by CD4+ T cells that enter the brain.

Fundamental to the transition from an innate to an adaptive immune response is the cell surface expression and antigen presentation by MHCII. Because CD74^{def} mice express lower baseline levels of cell surface MHCII compared to wild type (WT) C57B16 (FIG. 3A), we tested to ensure that the MHCII from these mice were still capable of responding to a pro-inflammatory insult, such as FPI, as such a response is important in the ability of APCs to present antigens to T cells. A trend indicating that CD74^{def} mice were capable of increased cell-surface expression of MHCII was observed at 24 hours after FPI (FIG. 3B). The results suggest that the CD74^{def} mice are capable of MHCII-mediated antigen presentation. Therefore, C74^{Def} mice are immune competent with respect to their ability to increase MHCII expression in response to FPI.

The data demonstrate that, consistent with the rat model of FPI from which the mouse model described herein is derived, our FPI mice are significantly impaired in terms of neurological and cognitive deficits, and that the cognitive deficits respond to CD74-targeted manipulations, including CLIP antagonism with CAP (FIG. 8A-C). The results support the efficacy of manipulating early and chronic PTBS.

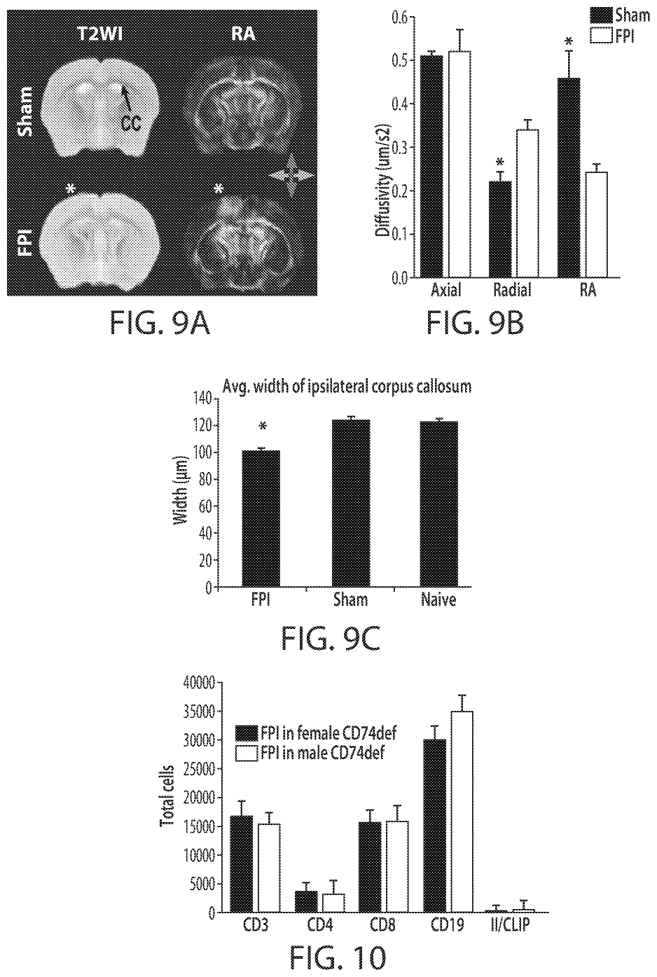

Diffuse axonal injury is well described in TBI models and in humans. Here, using diffusion tensor imaging (DTI), white matter loss has been shown at 30 days after our mouse FPI model (FIG. 9).

The CDC reports that females represent ˜⅖^{th} of all TBIs^{1/2}. Thus, female mice have been included to better model gender-specific outcomes. We examined this issue using male and female littermates from our CD74^{def} mouse colony. No significant gender effects were found on levels of immune cell types after FPI (FIG. 10).

### REFERENCES

1. C.f.D.C.a. Report to Congress on Traumatic Brain Injury in the United States: Epidemiology and Rehabilitation. in Prevention (National Center for Injury Prevention and Control, Atlanta, 2015).
2. C.f.D.C.a. Injury Prevention and Control: Traumatic Brain Injury & Concussion. Prevention (2016).
3. Zhang, Z., et al. Human traumatic brain injury induces autoantibody response against glial fibrillary acidic protein and its breakdown products. PLoS One 9, e92698 (2014).
4. Tobin, R. P., et al. Traumatic brain injury causes selective, CD74-dependent peripheral lymphocyte activation that exacerbates neurodegeneration. Acta Neuropathol Commun 2, 143 (2014).
5. Yang, D. B., et al. Serum macrophage migration inhibitory factor concentrations correlate with prognosis of traumatic brain injury. Clin Chim Acta 469, 99-104 (2017).
6. Heber-Katz, E., et al. Contribution of antigen-presenting cell major histocompatibility complex gene products to the specificity of antigen-induced T cell activation. J Exp Med 155, 1086-1099 (1982).
7. Maier, M., et al. Altered gene expression patterns in dendritic cells after severe trauma: implications for systemic inflammation and organ injury. Shock 30, 344-351 (2008).
8. Huttl, S., et al. Substrate determinants of signal peptide peptidase-like 2a (SPPL2a)-mediated intramembrane proteolysis of the invariant chain CD74. Biochem J 473, 1405-1422 (2016).
9. Huttl, S., et al. Processing of CD74 by the Intramembrane Protease SPPL2a Is Critical for B Cell Receptor Signaling in Transitional B Cells. J Immunol 195, 1548-1563 (2015).
10. Karin, M. & Ben-Neriah, Y. Phosphorylation meets ubiquitination: the control of NF-[kappa]B activity. Annu Rev Immunol 18, 621-663 (2000).
11. Lawrence, T. The nuclear factor NF-kappaB pathway in inflammation. Cold Spring Harb Perspect Biol 1, a001651 (2009).
12. Clausen, F., Lorant, T., Lewen, A. & Hillered, L. T lymphocyte trafficking: a novel target for neuroprotection in traumatic brain injury. J Neurotrauma 24, 1295-1307 (2007).
13. Yan, E. B., et al. Post-traumatic hypoxia is associated with prolonged cerebral cytokine production, higher serum biomarker levels, and poor outcome in patients with severe traumatic brain injury. J Neurotrauma 31, 618-629 (2014).
14. Cox, A. L., et al. An investigation of auto-reactivity after head injury. J Neuroimmunol 174, 180-186 (2006).
15. Blum, J. S., Wearsch, P. A. & Cresswell, P. Pathways of antigen processing. Annu Rev Immunol 31, 443-473 (2013).
16. Beswick, E. J., et al. Helicobacter pylori-induced IL-8 production by gastric epithelial cells up-regulates CD74 expression. J Immunol 175, 171-176 (2005).
17. Shi, X., et al. CD44 is the signaling component of the macrophage migration inhibitory factor-CD74 receptor complex. Immunity 25, 595-606 (2006).
18. Starlets, D., et al. Cell-surface CD74 initiates a signaling cascade leading to cell proliferation and survival. Blood 107, 4807-4816 (2006).
19. Shimabukuro-Vornhagen, A., et al. Antigen-presenting human B cells are expanded in inflammatory conditions. J Leukoc Biol 101, 577-587 (2017).
20. Basha, G., et al. A CD74-dependent MHC class I endolysosomal cross-presentation pathway. Nat Immunol 13, 237-245 (2012).
21. McDevitt, H. O. The role of H-2 I region genes in regulation of the immune response. J Immunogenet 8, 287-295 (1981).
22. Nakagawa, T. Y. & Rudensky, A. Y. The role of lysosomal proteinases in MHC class II-mediated antigen processing and presentation. Immunol Rev 172, 121-129 (1999).
23. Ankeny, D. P., Guan, Z. & Popovich, P. G. B cells produce pathogenic antibodies and impair recovery after spinal cord injury in mice. J Clin Invest 119, 2990-2999 (2009).
24. Schori, H., Lantner, F., Shachar, I. & Schwartz, M. Severe immunodeficiency has opposite effects on neuronal survival in glutamate-susceptible and -resistant mice: adverse effect of B cells. J Immunol 169, 2861-2865 (2002).
25. Schori, H., Shechter, R., Shachar, I. & Schwartz, M. Genetic manipulation of CD74 in mouse strains of different backgrounds can result in opposite responses to central nervous system injury. J Immunol 178, 163-171 (2007).
26. McKee, C. A. & Lukens, J. R. Emerging Roles for the Immune System in Traumatic Brain Injury. Front Immunol 7, 556 (2016).
27. Truettner, J. S., Suzuki, T. & Dietrich, W. D. The effect of therapeutic hypothermia on the expression of inflammatory response genes following moderate traumatic brain injury in the rat. Brain Res Mol Brain Res 138, 124-134 (2005).
28. Nakagawa, T. Y., et al. Impaired invariant chain degradation and antigen presentation and diminished collagen-induced arthritis in cathepsin S null mice. Immunity 10, 207-217 (1999).
29. Haves-Zburof, D., et al. Cathepsins and their endogenous inhibitors cystatins: expression and modulation in multiple sclerosis. J Cell Mol Med 15, 2421-2429 (2011).
30. Slavin, A. J., et al. Requirement for endocytic antigen processing and influence of invariant chain and H-2M deficiencies in CNS autoimmunity. J Clin Invest 108, 1133-1139 (2001).
31. Bruce-Keller, A. J., et al. Gender and estrogen manipulation do not affect traumatic brain injury in mice. J Neurotrauma 24, 203-215 (2007).
32. Murphy, K., Travers, P., Walport, M. & Janeway, C. Janeway's immunobiology, (Garland Science, New York, 2008).
33. Collins, A. M. & Jackson, K. J. A Temporal Model of Human IgE and IgG Antibody Function. Front Immunol 4, 235 (2013).
34. Elliot, L. N., Lloyd, A. R., Ziegler, J. B. & Ffrench, R. A. Protective immunity against hepatitis C virus infection. Immunol Cell Biol 84, 239-249 (2006).
35. Anderson, S. J. & Coleclough, C. Regulation of CD4 and CD8 expression on mouse T cells. Active removal from the cell surface by two mechanisms. J Immunol 151, 5123-5134 (1993).
36. Chen, S. K., et al. Hematopoietic origin of pathological grooming in Hoxb8 mutant mice. Cell 141, 775-785 (2010).
37. Filiano, A. J., et al. Unexpected role of interferon-gamma in regulating neuronal connectivity and social behaviour. Nature 535, 425-429 (2016).
38. Chang, K., et al. Clinical evaluation of youth with pediatric acute-onset neuropsychiatric syndrome (PANS): recommendations from the 2013 PANS Consensus Conference. J Child Adolesc Psychopharmacol 25, 3-13 (2015).
39. Mukherjee, S., Zeitouni, S., Cavarsan, C. F. & Shapiro, L. A. Increased seizure susceptibility in mice 30 days after fluid percussion injury. Front Neurol 4, 28 (2013).
40. Robinson, C., Apgar, C. & Shapiro, L. A. Astrocyte Hypertrophy Contributes to Aberrant Neurogenesis after Traumatic Brain Injury. Neural Plast 2016, U.S. Pat. No. 1,347,987 (2016).
41. Arisi, G. M., et al. Astrocyte Alterations in the Hippocampus Following Pilocarpine-induced Seizures in Aged Rats. Aging Dis 2, 294-300 (2011).
42. Shapiro, L. A., Perez, Z. D., Foresti, M. L., Arisi, G. M. & Ribak, C. E. Morphological and ultrastructural features of Ibal-immunolabeled microglial cells in the hippocampal dentate gyrus. Brain Res 1266, 29-36 (2009).
43. Shapiro, L. A., Wang, L. & Ribak, C. E. Rapid astrocyte and microglial activation following pilocarpine-induced seizures in rats. Epilepsia 49 Suppl 2, 33-41 (2008).
44. Sun, S. W., Neil, J. J. & Song, S. K. Relative indices of water diffusion anisotropy are equivalent in live and formalin-fixed mouse brains. Magn Reson Med 50, 743-748 (2003).
45. Mukherjee, S., Katki, K., Arisi, G. M., Foresti, M. L. & Shapiro, L. A. Early TBI-Induced Cytokine Alterations are Similarly Detected by Two Distinct Methods of Multiplex Assay. Front Mol Neurosci 4, 21 (2011).
46. Bucala, R. & Shachar, I. The integral role of CD74 in antigen presentation, MIF signal transduction, and B cell survival and homeostasis. Mini Rev Med Chem 14, 1132-1138 (2014).
47. Pierre, P. & Mellman, I. Developmental regulation of invariant chain proteolysis controls MHC class II trafficking in mouse dendritic cells. Cell 93, 1135-1145 (1998).
48. Biragyn, A., Aliseychik, M. & Rogaev, E. Potential importance of B cells in aging and aging-associated neurodegenerative diseases. Semin Immunopathol 39, 283-294 (2017).
49. De Virgilio, A., et al. Parkinson's disease: Autoimmunity and neuroinflammation. Autoimmun Rev 15, 1005-1011 (2016).
50. Dalakas, M. C. B cells as therapeutic targets in autoimmune neurological disorders. Nat Clin Pract Neurol 4, 557-567 (2008).
51. Newell, M. K., et al. TLR-mediated B cell activation results in ectopic CLIP expression that promotes B cell-dependent inflammation. J Leukoc Biol 88, 779-789 (2010).
52. Collison, L. W. & Vignali, D. A. In vitro Treg suppression assays. Methods Mol Biol 707, 21-37 (2011).
53. Santarsieri, M., Kumar, R. G., Kochanek, P. M., Berga, S. & Wagner, A. K. Variable neuroendocrine-immune dysfunction in individuals with unfavorable outcome after severe traumatic brain injury. Brain Behav Immun 45, 15-27 (2015).
54. Jacquin, M. F., et al. In DRG11 knock-out mice, trigeminal cell death is extensive and does not account for failed brainstem patterning. J Neurosci 28, 3577-3585 (2008).
55. Ribak, C. E., et al. Seizure-induced formation of basal dendrites on granule cells of the rodent dentate gyrus. in Jasper's Basic Mechanisms of the Epilepsies (eds. th, et al.) (Bethesda (Md.), 2012).
56. van Goethem, N. P., Schreiber, R., Newman-Tancredi, A., Varney, M. & Prickaerts, J. Divergent effects of the ‘biased’ 5-HT1 A receptor agonists F15599 and F13714 in a novel object pattern separation task. Br J Pharmacol 172, 2532-2543 (2015).
57. Costa, A. A., Morato, S., Roque, A. C. & Tinos, R. A computational model for exploratory activity of rats with different anxiety levels in elevated plus-maze. J Neurosci Methods 236, 44-50 (2014).
58. Hattiangady, B., Kuruba, R. & Shetty, A. K. Acute Seizures in Old Age Leads to a Greater Loss of CA1 Pyramidal Neurons, an Increased Propensity for Developing Chronic TLE and a Severe Cognitive Dysfunction. Aging Dis 2, 1-17 (2011).
59. Hattiangady, B. & Shetty, A. K. Neural stem cell grafting counteracts hippocampal injury-mediated impairments in mood, memory, and neurogenesis. Stem Cells Transl Med 1, 696-708 (2012).
60. Critchley, H., et al. Explicit and implicit neural mechanisms for processing of social information from facial expressions: a functional magnetic resonance imaging study. Hum Brain Mapp 9, 93-105 (2000).
61. Critchley, H. D., et al. The functional neuroanatomy of social behaviour: changes in cerebral blood flow when people with autistic disorder process facial expressions. Brain 123 (Pt 11), 2203-2212 (2000).
62. Vegeto, E., et al. Estrogen receptor-alpha mediates the brain antiinflammatory activity of estradiol. Proc Natl Acad Sci USA 100, 9614-9619 (2003).

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of treating a subject having a traumatic brain disorder (TBI), comprising:
administering to a subject having a TBI a macrophage migration inhibition factor (MIF) antagonist in an effective amount to treat the TBI.

2. The method of claim 1, wherein the MIF antagonist is an anti-MIF antibody.

3. The method of claim 2, wherein the anti-MIF antibody is a humanized anti-MIF antibody (Imalumab).

4. The method of claim 1, wherein the MIF antagonist is a small molecule MIF antagonist.

5. The method of claim 4, wherein the small molecule MIF antagonist has one or more of the following properties: oral bioavailability, bioavailability, and blood brain barrier permeability.

6. The method of claim 4, wherein the MIF antagonist is selected from the group consisting of ISO1, MIF098, MIF139, MIF108, MIF046, MIFhom, and MIFacid.

7. The method of any one of claims 1-6, further comprising administering to the subject an isolated MHC class II specific CLIP inhibitor.

8. The method of claim 7, wherein CLIP inhibitor is a synthetic peptide.

9. The method of claim 8, wherein the synthetic peptide comprises X₁RX₂X₃X₄X₅LX₆X₇(SEQ ID NO: 1), wherein each X is an amino acid, wherein R is Arginine, L is Leucine and wherein at least one of X₂ and X₃ is Methionine.

10. The method of claim 9, wherein X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine

11. The method of claim 9, wherein the peptide further comprises 1-5 amino acids at the N and/or C terminus.

12. The method of claim 9, wherein the peptide comprises FRIM X₄VLX₆S (SEQ ID NO: 3), wherein X₄ and X₆ are any amino acid.

13. The method of claim 9, wherein the peptide comprises FRIMAVLAS (SEQ ID NO: 4).

14. The method of claim 9, wherein the peptide has 9-20 amino acids.

15. The method of claim 9, wherein the peptide is non-cyclic.

16. The method of claim 7, wherein the CLIP inhibitor is an siRNA.

17. The method of any one of claims 1-15, further comprising administering to the subject a CD74 cleavage inhibitor.

18. The method of claim 17, wherein the CD74 cleavage inhibitor is a SPP2La cleavage inhibitor.

19. The method of claim 18, wherein the SPP2La cleavage inhibitor is Brefeldin A.

20. The method of claim 17, wherein the CD74 cleavage inhibitor is a cathepsin S inhibitor, optionally Cystatin S.

21. The method of claim 17, wherein the CD74 cleavage inhibitor is selected from the group consisting of CST1, CST2, CST3 (cystatin C), CST4, CST5, CST6, CST7, CST8, CST9, CST11, CSTA (cystatin A), CSTB (cystatin B), histidine-rich glycoprotein (HRG), fetuins, cystatin-related protein, Spp24, cystatin-related epididymal spermatogenic (CRES) protein, Brefeldin A, Bortezomib (PS341), cystatin S, Z-FF-FMK, Z-FY-CHO, Z-FY(tBu)-DMK, E-64, E-64C, and E-64D.

22. The method of any one of claims 1-21, wherein the TBI is post-traumatic brain injury syndromes (PTS).

23. The method of any one of claims 1-21, wherein the subject is treated within a week of the TBI.

24. The method of any one of claims 1-21, wherein the subject is treated within 24 hours of the TBI.

25. The method of any one of claims 1-21, wherein the subject is treated within 8 hours of the TBI.

26. The method of any one of claims 1-21, wherein the subject is treated within 2 hours of the TBI.

27. The method of any one of claims 1-21, wherein the subject is treated within 30 minutes of the TBI.

28. The method of any one of claims 1-27, further comprising using a head injury monitor to detect the presence of a head injury.

29. The method of claim 28, wherein the head injury monitor is selected from the group consisting of a CheckLight™ device or a X-Patch™.

30. The method of any one of claims 1-29, wherein the subject is administered at least 2 doses of MIF antagonist.

31. The method of any one of claims 1-29, wherein the subject is administered at least 3 doses of MIF antagonist.

32. The method of any one of claims 1-29, wherein the MIF antagonist is administered to the subject weekly.

33. A method of treating a subject having a traumatic brain disorder (TBI), comprising:
administering to a subject having a TBI a CD74 cleavage inhibitor in an effective amount to treat the TBI.

34. The method of claim 33, wherein the CD74 cleavage inhibitor is a SPP2La cleavage inhibitor, optionally Brefeldin A.

35. The method of claim 33, wherein the CD74 cleavage inhibitor is a cathepsin S inhibitor.

36. The method of claim 33, wherein the CD74 cleavage inhibitor is selected from the group consisting of Cystatin S, CST1, CST2, CST3 (cystatin C), CST4, CST5, CST6, CST7, CST8, CST9, CST11, CSTA (cystatin A), CSTB (cystatin B), histidine-rich glycoprotein (HRG), fetuins, cystatin-related protein, Spp24, cystatin-related epididymal spermatogenic (CRES) protein, Brefeldin A, Bortezomib (PS341), cystatin S, Z-FF-FMK, Z-FY-CHO, Z-FY(tBu)-DMK, E-64, E-64C, and E-64D.

37. The method of any one of claims 33-36, further comprising administering to the subject an isolated MHC class II specific CLIP inhibitor.

38. The method of claim 37, wherein CLIP inhibitor is a synthetic peptide.

39. The method of claim 38, wherein the synthetic peptide comprises X₁RX₂X₃X₄X₅LX₆X₇(SEQ ID NO: 1), wherein each X is an amino acid, wherein R is Arginine, L is Leucine and wherein at least one of X₂ and X₃ is Methionine.

40. The method of claim 39, wherein X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine

41. The method of claim 39, wherein the peptide further comprises 1-5 amino acids at the N and/or C terminus.

42. The method of claim 39, wherein the peptide comprises FRIM X₄VLX₆S (SEQ ID NO: 3), wherein X₄ and X₆ are any amino acid.

43. The method of claim 39, wherein the peptide comprises FRIMAVLAS (SEQ ID NO: 4).

44. The method of claim 39, wherein the peptide has 9-20 amino acids.

45. The method of claim 39, wherein the peptide is non-cyclic.

46. The method of claim 37, wherein the CLIP inhibitor is an siRNA.

47. The method of any one of claims 33-45, further comprising administering to the subject a macrophage migration inhibition factor (MIF) antagonist.

48. The method of claim 47, wherein the MIF antagonist is an anti-MIF antibody.

49. The method of claim 48, wherein the anti-MIF antibody is a humanized anti-MIF antibody (Imalumab).

50. The method of claim 47, wherein the MIF antagonist is a small molecule MIF antagonist.

51. The method of claim 50, wherein the small molecule MIF antagonist has one or more of the following properties: oral bioavailability, bioavailability, and blood brain barrier permeability.

52. The method of claim 50, wherein the MIF antagonist is selected from the group consisting of ISO1, MIF098, MIF139, MIF108, MIF046, MIFhom, and MIFacid.

53. The method of any one of claims 33-52, wherein the TBI is post-traumatic brain injury syndromes (PTS).

54. The method of any one of claims 33-53, wherein the subject is treated within a week of the TBI.

55. The method of any one of claims 33-53, wherein the subject is treated within 24 hours of the TBI.

56. A method of treating a subject at risk of having a seizure, comprising:
administering to a subject at risk of having a seizure an isolated MHC class II specific CLIP inhibitor in an effective amount to treat the subject.

57. The method of claim 56, wherein the CLIP inhibitor is a synthetic peptide.

58. The method of claim 57, wherein the synthetic peptide comprises X₁RX₂X₃X₄X₅LX₆X₇(SEQ ID NO: 1), wherein each X is an amino acid, wherein R is Arginine, L is Leucine and wherein at least one of X₂ and X₃ is Methionine.

59. The method of claim 58, wherein X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine

60. The method of claim 8, wherein the peptide further comprises 1-5 amino acids at the N and/or C terminus.

61. The method of claim 58, wherein the peptide comprises FRIM X₄VLX₆S (SEQ ID NO: 3), wherein X₄ and X₆ are any amino acid.

62. The method of claim 58, wherein the peptide comprises FRIMAVLAS (SEQ ID NO: 4).

63. The method of any one of claims 56-62, wherein the peptide has 9-20 amino acids.

64. The method of claim 58, wherein the peptide is non-cyclic.

65. The method of claim 56, wherein the CLIP inhibitor is an siRNA.

66. The method of any one of claims 56-65, further comprising administering to the subject a CD74 cleavage inhibitor.

67. The method of any one of claims 56-65, further comprising administering to the subject a macrophage migration inhibition factor (MIF) antagonist.

68. The method of claim 67, wherein the MIF antagonist is an anti-MIF antibody.

69. The method of claim 68, wherein the anti-MIF antibody is a humanized anti-MIF antibody (Imalumab).

70. The method of claim 67, wherein the MIF antagonist is a small molecule MIF antagonist.

71. The method of claim 70, wherein the MIF antagonist is selected from the group consisting of ISO1, MTF098, MTF139, MIF108, MTF046, MIFhom, and MTFacid


SEQUENCE LISTING (18 sequences)

SEQ ID NO: 1 - protein, 9 aa, Artificial Sequence
  Synthetic Polynucleotide
  MISC_FEATURE (1)..(1): Xaa is Ala, Phe, Met, Leu, Ile, Val, Pro or Trp
  MISC_FEATURE (3)..(3): Xaa is Ala, Phe, Met, Leu, Ile, Val, Pro or Trp
  MISC_FEATURE (4)..(4): Xaa is Ala, Phe, Met, Leu, Ile, Val, Pro or Trp
  MISC_FEATURE (5)..(5): Xaa can be any naturally occurring amino acid
  MISC_FEATURE (6)..(6): Xaa is Ala, Phe, Met, Leu, Ile, Val, Pro or Trp
  MISC_FEATURE (8)..(8): Xaa can be any naturally occurring amino acid
  MISC_FEATURE (9)..(9): Xaa is Ala, Cys, Thr, Ser, Gly, Asn, Glyn, or Tyr
XRXXXXLXX

SEQ ID NO: 2 - protein, 9 aa, Artificial Sequence
  Synthetic Polynucleotide
MRMATPLLM

SEQ ID NO: 3 - protein, 9 aa, Artificial Sequence
  Synthetic Polynucleotide
  MISC_FEATURE (5)..(5): Xaa can be any naturally occuring amino acid
  MISC_FEATURE (8)..(8): Xaa can be any naturally occuring amino acid
FRIMXVLXS

SEQ ID NO: 4 - protein, 9 aa, Artificial Sequence
  Synthetic Polynucleotide
FRIMAVLAS

SEQ ID NO: 5 - protein, 9 aa, Artificial Sequence
  Synthetic Polynucleotide
IRIMATLAI

SEQ ID NO: 6 - protein, 9 aa, Artificial Sequence
  Synthetic Polynucleotide
FRIMAVLAI

SEQ ID NO: 7 - protein, 9 aa, Artificial Sequence
  Synthetic Polynucleotide
IRIMAVLAS

SEQ ID NO: 8 - protein, 115 aa, Artificial Sequence
  Synthetic Polypeptide
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 9 - RNA, 19 nt, Artificial Sequence
  Synthetic Polynucleotide
gguaguaauuagaacaaaa

SEQ ID NO: 10 - RNA, 19 nt, Artificial Sequence
  Synthetic Polynucleotide
gguucacauuagaauaaaa

SEQ ID NO: 11 - RNA, 19 nt, Artificial Sequence
  Synthetic Polynucleotide
gaacaaaaaaaaaaaaaaa

SEQ ID NO: 12 - RNA, 19 nt, Artificial Sequence
  Synthetic Polynucleotide
caaaaaaaaaaaaaaaaaa

SEQ ID NO: 13 - RNA, 19 nt, Artificial Sequence
  Synthetic Polynucleotide
agaacaaaaaaaaaaaaaa

SEQ ID NO: 14 - RNA, 19 nt, Artificial Sequence
  Synthetic Polynucleotide
acaaaaaaaaaaaaaaaaa

SEQ ID NO: 15 - RNA, 19 nt, Artificial Sequence
  Synthetic Polynucleotide
guaauuagaacaaaaaaaa

SEQ ID NO: 16 - RNA, 19 nt, Artificial Sequence
  Synthetic Polynucleotide
caugguucacauuagaaua

SEQ ID NO: 17 - RNA, 19 nt, Artificial Sequence
  Synthetic Polynucleotide
guaguaauuagaacaaaaa

SEQ ID NO: 18 - RNA, 19 nt, Artificial Sequence
  Synthetic Polynucleotide
ggcuuuucuagccuauuua